(19) United States
(12) Patent Application Publication
(10) Pub. No.: US 2020/0078185 A1
(43) Pub. Date: Mar. 12, 2020

(54) First Metatarsal Hemi-Arthroplasty Implant

(71) Applicants: Richard MARKS, Mobile, AL (US); Rachelle YUSUFBEKOV, Jupiter, FL (US)

(72) Inventors: Richard MARKS, Mobile, AL (US); Rachelle YUSUFBEKOV, Jupiter, FL (US)

(21) Appl. No.: 16/463,891

(22) PCT Filed: Nov. 28, 2017
(86) PCT No.: PCT/US17/63361

§ 371 Date: May 24, 2019

Related U.S. Application Data
(60) Provisional application No. 62/428,858, filed on Dec. 1, 2016.

(51) Int. Cl. A61F 2/42; A61F 2/30

(52) U.S. Cl. CPC A61F 2/4225; A61F 2/30744; A61F 2002/4619; A61F 2002/30884; A61F 2002/4235; A61F 2002/30841

(57) ABSTRACT

A first or lesser metatarsal hemi-arthroplasty implant and system for performing a hemi-arthroplasty of the first or lesser metatarsal phalangeal joint is described. The implant preferably includes a concave head and a proximal stem wherein the proximal stem may include one or more fins thereon and one or more peripherally spaced spike members to limit the rotational movement of the implant once it is inserted. A pair of notched members are provided along the periphery of the implant to assist in the removal of the implant in the event of surgical failure. An implant template is provided that includes a central opening, with spaced holes and an etched surface to facilitate the alignment and positioning of the implant.

### PRIORITY CLAIM

The present application is a continuation-in-part of U.S. Ser. No. 62/428,858 filed on Dec. 1, 2016

### FIELD OF THE INVENTION

The present invention relates generally to the field of orthopaedic and podiatric surgery. More particularly, the present invention relates to the treatment of arthritis of the big toe joint or limited dorsiflexion (hallux rigidus) of the first metatarsophalangeal (MTP) joint as well as surgical treatment of the lesser metatarsophalangeal joints, because of dorsal osteophyte impingement or treatment of arthritis of the first MTP and lesser MTP joints.

### BACKGROUND

Arthritis of the big toe is the most common arthritic condition of the foot and is second only to bunions (hallux valgus) as a condition associated with the big toe. The true cause of hallux rigidus is not known although, several risk factors such as an abnormally long or elevated first foot bone (metatarsal), differences in foot anatomy, prior traumatic injury to the big toe or family history are believed to be contributing factors. Hallux rigidus is typically diagnosed by physical examination of the joint by a physician. This examination includes manipulation of the metatarsophalangeal (MTP) joint and examination of the foot for evidence of bone spurs. X-rays may then be used to help understand the extent of joint degeneration and to show the location and size of bone spurs.

Treatment of hallux rigidus typically consists of non-surgical therapy that includes anti-inflammatory medications, heat or ice, orthotics and injections. Surgical options are determined by the failure of the non-surgical therapy and the extent of the arthritis located in the MTP joint. Common surgical options for hallux rigidus and arthritis of the first MTP joint include decompression (cheilectomy), partial joint resection (resection arthroplasty), fusion (arthrodesis) of the 1st MTP joint, joint replacement and hemi-arthroplasty (partial joint replacement). Cheilectomy involves shaving the bone spur to allow more room for the toe to bend. Resection arthroplasty involves the cleaning of the arthritis and bone spurs from the MTP joint, combined with resection of the base of the proximal phalanx and then sewing the tissue around the joint capsule. The presence of arthritic complaints may fail to alleviate patients' symptoms following cheilectomy, and resection arthroplasty has an unacceptably high rate of failure, loss of function and secondary deformity. First MTP joint fusion limits motion of the joint, and many active individuals report significant limitations of activity post-fusion. Existing joint replacement and hemiarthroplasty products/devices for treatment of first MTP joint arthritis commonly disrupt the surrounding soft tissues, creating loss of normal function of the first MTP joint, and resulting abnormal biomechanics and gait. Excessive bone resection can result in shortening of the hallux, loosening of implant fixation and implant failure. Revision or replacement of a failed first MTP implant or hemiarthroplasty thus becomes more complex due to bone loss, change in joint length or configuration and disruption of the surrounding soft tissues. The lesser MTP joints may also, be affected by arthritic changes, though to a lesser degree. Common treatment consists of either bone resection, or resculpting of the metatarsal heads with a rongeur or reamer. This treatment can result in shortening of the metatarsal and respective toe, or transfer metatarsalgia as a result of the shortening. Therefore, it is important to maintain the same or similar length of the toe and joint as it was prior to the surgery.

Examples of various hemi-arthroplasty (partial joint) implants are disclosed in U.S. Patent Application 20040230303A1 filed by Saunders, U.S. Patent Application No. 20080051912 filed by Hollawell and U.S. Patent Application No. 20120259419A1 filed by Brown et. al. In each of these devices, a generally centrally positioned stem or anchor is fixated into the bone and a generally concave bearing or contact surface is provided. U.S. Pat. No. 9,044,332 granted to Goswami et. al., discloses numerous configurations of implantable devices for replacing all or a portion of the MTP joint. U.S. Patent Application No. 20100262254 filed by Lawrence et. al., disclosed a MTP implant that is oriented at an angle between approximately 45 and 75 degrees relative to the bone engaging surface. Similarly, PCT Publication No. WO 2011090711A1 filed by Beckendorf et. al, discloses a resurfacing implant including a head having a convex outer surface overlaying a concave inner surface. The implant also includes a stem extending from the inner surface such that the edge of the head surrounds the stem and overhangs a portion of the stem leading up to the inner surface. The device disclosed in the Beckendorf et. al publication appears to be similar to the ENCOMPASS metatarsal resurfacing implant sold by Osteomed, Inc. of Addison, Tex., USA. U.S. Patent Application No. 20120215320A1 discloses an implant for metatarsal hemiarthroplasty having first and second surfaces wherein the concave surface may include a pair of projection members which extend into the articular head of the bone.

There is a need for a surgical alternative that alleviates arthritic symptoms, while maintaining normal anatomy, biomechanics and motion of the first MTP joint. Ideally, a resurfacing option would limit bone resection and maintain soft tissue integrity, while allowing for the option of joint fusion if the hemi-arthroplasty is not successful and a full joint revision is necessary. The metatarsal hemi-arthroplasty of the present invention maintains metatarsal bone stock and length, while maintaining the integrity of the sesamoid complex, plantar plate, and collateral ligaments of the affected MTP joint. The implant is available to users in multiple sizing options for use in the 1st MTP or lesser MTP joints, and can be converted to a fusion procedure of the 1st MTP joint without the need for structural bone grafting material if revision is required. Additionally, the use of a single stem member with one or more spike members reduces the impact to the metatarsal head of the patient and minimizes the amount of bone that needs to be removed for implant placement. Because the amount of bone that is removed is minimized, if the implant fails, it is easier for the surgeon to perform a full joint revision.

### SUMMARY

The present invention is directed to a metatarsal implant for a foot. The metatarsal implant is designed to be inserted into a stem aperture formed in the metatarsal bone of the foot and to form a joint with the proximal phalanx of the great toe. The metatarsal implant generally includes a head portion and a stem. The head includes an articular joint engaging surface and an opposed, bone engaging surface. The joint engaging surface is adapted to engage the proximal phalanx. The stem extends into the metatarsal bone of the foot. It is anticipated that the implant

may be used in both the 1st MTP and the lesser MTP joints. The bone engaging surface of the implant may be comprised of a smooth, roughened or porous surface.

The head of the present invention preferably includes a tapering thickness from the central portion to the periphery thereof. The use of the tapering thickness reduces the amount of bone that needs to be removed for proper implant placement. The outer surface of the head is also generally semi-circularly shaped and includes at least a pair of indents therein to facilitate the removal thereof in the event that the procedure is not successful, and a full joint implant or fusion is necessary. The interior surface of the head includes the stem extending therefrom. The stem may be a conical member that has a constant thickness, decreases in thickness along the length thereof or it may include one or more fin members to limit the rotational movement thereof when the implant is placed in the metatarsal bone. Additionally, the interior of the head includes one or more spikes spaced laterally from the centrally located stem. In a preferred form, three spikes are evenly spaced from the interior of the head in a generally peace sign shaped configuration. The spikes are configured to extend into the metatarsal bone to resist rotational movement of the implant relative to the metatarsal bone. The combination of the stem member with one or more spike members allows the thickness of the stem member to be reduced while the spike members and/or the use of fin members on the stem member resist rotational movement of the implant during use. This also allows the surgeon to remove less bone from the metatarsal head during the procedure. The ability to reduce bone removal allows the surgeon to maintain the symmetry of the MTP joint with less bone loss and also provides a larger distal end of the metatarsal bone in the event of implant failure.

It will be understood by those skilled in the art that one or more aspects of this invention can meet certain objectives, while one or more other aspects can lead to certain other objectives. Other objects, features, benefits and advantages of the present invention will be apparent in this summary and descriptions of the disclosed embodiment, and will be readily apparent to those skilled in the art. Such objects, features, benefits and advantages will be apparent from the above as taken in conjunction with the accompanying figures and all reasonable inferences to be drawn therefrom.

### BRIEF DESCRIPTION OF THE DRAWINGS

FIG. 1 is a side perspective view of a portion of a foot and a metatarsal implant device having features of the present invention;

FIG. 2 is front perspective view showing the implant device installed on the head of the metatarsal bone;

FIG. 3 is a side perspective view of the implant device having features of the present invention;

FIG. 4 is a top perspective view of the implant device showing the head surface of the implant;

FIG. 5 is a rear view of an alternate form of the implant device showing the stem and spikes of the implant;

FIG. 6 is a partial cross-sectional view of the implant device shown in FIG. 5;

FIG. 7 is a cross sectional view of an alternate form of the implant device of the present invention;

FIG. 8A is a side view, partially in cross section, of an alternate embodiment of an implant device of the present invention;

FIG. 8B is a cross sectional view of the embodiment of FIG. 8A;

FIG. 9A is a bottom view of an alternate embodiment of the implant device of the present invention;

FIG. 9B is a cross sectional view of the embodiment of FIG. 9A;

FIG. 10A is a bottom view of another alternate embodiment of the implant of the present invention;

FIG. 10B is a cross sectional view of the embodiment of FIG. 10A.

FIG. 11A is a bottom perspective view of another alternate embodiment of the present invention;

FIG. 11B is a cross sectional view of the embodiment of FIG. 11A;

FIG. 11C is a side view of the embodiment of FIG. 11A

FIG. 12A is a bottom perspective view of another alternate embodiment of the present invention;

FIG. 12B is a side view of the embodiment of FIG. 12A; and

FIG. 12C is a cross sectional view of the embodiment of FIG. 12A.

### DETAILED DESCRIPTION

The first metatarsal hemi-arthroplasty system of the present invention includes implants and instrumentation to perform a resurfacing procedure of the first MTP or lesser MTP joints. The system includes instrumentation to assist in the placement of the implant, including but not limited to instrumentation to guide the implant to the desired location. Such instrumentation may include reamers, routers, rasps, broaches, saws, guide pins and guide wires as well as other placement or guidance tools specific to arthroplasty procedures. Additional tooling to establish pilot holes for the assistance of implant placement may be used.

The system also includes instrumentation for the preparation of the metatarsal head to receive the anatomy conserving implant. Bone preparation instrumentation may include, but is not limited to a reamer, saw, rasp and/or alternative bone preparation devices. Alternative bone preparation devices may include those devices used in arthroplasty procedures to resect tissue (soft and hard) to prepare the surgical site to receive the implant.

The system also includes instrumentation for implant sizing and instrumentation for inserting the implant into the metatarsal head. This may include implant embodiments for both manual insertion of the implant or implant insertion using instrumentation specific tools for implantation.

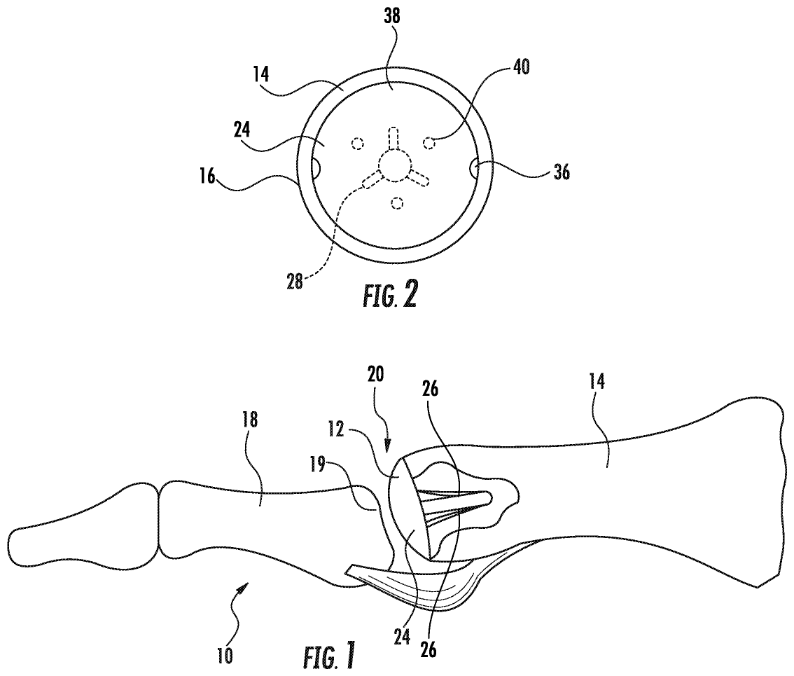

FIGS. 1 and 2 show the foot 10 of a patient having a metatarsal implant shown generally as component 12. The foot 10 includes a metatarsal bone 14 having a distal end 16 on which the implant 12 is attached. The foot 10 also includes a proximal phalanx bone 18 having a proximal end 19 which is positioned adjacent to the implant 12 and the distal end 16 of the metatarsal bone 14 to form the components of the metatarsal phalangeal joint 20.

The implant 12 is designed to be anatomy conserving and is comprised of a proximal stem 22 and a concave head 24. The proximal stem 22 of the implant 12 is implanted into a stem aperture 26 that is drilled into the distal end 16 of the metatarsal bone 14. As shown, the proximal stem 22 may have a generally conical or circular in cross sectional shape that decreases in width as it extends away from the concave head 24. In an alternate form of the present invention, the proximal stem 22 may include one or more flange like fin members 28 that extend laterally and taper inwardly from the proximal stem 22 to the end 23 thereof. As shown, the fin members 28 are wider and extend outwardly further near the proximal stem adjacent to the concave head 24 than the width of the fin members 28 adjected to the end 23 of the proximal stem 22. Alternatively, the proximal stem 22 may be comprised of a roughened or porous surface, a threaded surface or smooth surface.

The distal end of the implant 12 is a generally concave shaped head member 24. The head member 24 includes a smooth exterior surface 30 that interfaces with the neighboring proximal phalanx bone 16 in the MTP joint 20. The concave head 24 is positioned generally adjacent to the proximal end 19 of the proximal phalanx bone 18 when the implant 12 is properly implanted onto the distal end 16 of the metatarsal bone 14. The head member 24 also includes an interior surface 32 (surface that interfaces with the metatarsal head) that may be comprised of a roughened, textured, or porous surface. Alternatively, a non-roughened surface or a non-porous surface may be used on the interior surface 32 of the implant 12 for an alternative implant embodiment. As shown, the exterior surface 30 and the interior surface 32 of the head member 24 preferably decrease in thickness from the center portion of the head member 24 to the periphery 34 thereof to minimize the amount of bone that must be removed during the procedure to properly seat the implant 12. Also, as shown and described more fully below, the periphery 32 of the head member 24 preferably includes one or more preferably laterally oriented notch members 36 to facilitate the removal of the implant if the doctor determines that the implant has failed, and a joint fusion or full joint implant is appropriate. Additionally, as shown, an etched member 38 is provided along the top edge thereof to assist in identifying the proper alignment of the implant 12 on the metatarsal bone 14.

The interior surface 32 of the head member 24 also includes a plurality of spike shaped members 40. The spike members 40 are spaced apart from the proximal stem 22 and each other. As shown in the drawings, the spike members 40 are preferably smaller and have a sharper profile than the proximal stem 22 to extend into the distal end 16 of the metatarsal bone 14. The spike members 40, in addition to the fin members 28 (if present), prevent rotational movement of the implant 12 once it is affixed to the distal end 16 of the metatarsal bone 14. FIGS. 2, 5 and 8-10 are illustrative of various alternate embodiments of the present invention. As shown, the implant 12 may include a proximal stem 22 that is tapered or of constant diameter. The stem may also have a plurality of fin members 28 arranged in a desired configuration such as three fins 28 arranged in a trident configuration preferably aligned at a 4, 8 and 12 clock orientations. The fin members may be of consistent width or may decrease in width near the end 23 of the proximal stern. One or more spike members 40 may also be included and in one configuration, the spike members 40 may be aligned at 2, 6 and 10 clock orientations. Alternately, two spike members 40 may be utilized at 2 and 10 o'clock configurations or a single spike may be aligned at a 6 or 12 o'clock configuration. The utilization of multiple fin members 28 and one or more spike members 40 provide added stability to the implant 12 to reduce the likelihood of rotational movement of the implant and therefore potential failure of the implant while minimizing the amount of bone removal required for proper implant placement.

The procedure for 1st MTP joint implantation is performed under regional anesthesia (ankle block) and intravenous sedation. The patient is placed supine on the operating room table, the foot and ankle are sterilely prepped to above the ankle. The limb is exsanguinated, and the procedure is performed typically with a tourniquet at the level of the ankle. A dorsal longitudinal incision is made starting at the distal ⅓ of the 1st metatarsal (MT), and extended distally to the proximal ⅓ of the proximal phalanx of the hallux. The EHL tendon is retracted laterally, and the capsule to the MTP joint sharply incised. Dissection is continued medially and laterally to allow exposure of the metatarsal head and base of the proximal phalanx. Sufficient exposure medially and laterally should allow full visualization of the inferior aspect of the 1st MT. Any bony eminence or osteophytes along the dorsal, medial and lateral aspect of the 1st MT are resected with a rongeur, chisel, and/or saw. Osteophytes at the dorsal base of the proximal phalanx are also resected with similar instrumentation. The sesamoid complex is mobilized with an elevator to maximize dorsiflexion of the 1st MTP joint. Although not described herein, a substantially similar surgical procedure is used for surgical procedures involving the lesser MTP joints.

A free reamer is used to initially determine the size the MT head and the corresponding implant size, as well as to assist with orientation for the guide pin insertion. The guide pin is inserted under power to a depth corresponding to the laser-etched line on the guide pin, and the position of the guide pin is checked fluoroscopically in both the frontal and lateral planes. The surgeon then chooses between the standard reamer sizes of 14, 16, 18, 20, 22, or 24. If the size of the MT head is between sizes the surgeon uses the reamer size that is undersized to avoid impingement of the implant. The reamer has a window to assess the depth of bone resection. Markings are located on the guide pin for 1-6 mm resection. There is preferably a hard stop at 6 mm to prevent the guide pin from being inserted too far. Reaming is then performed to remove the cartilage and subchondral bone down to the bleeding cancellous bone.

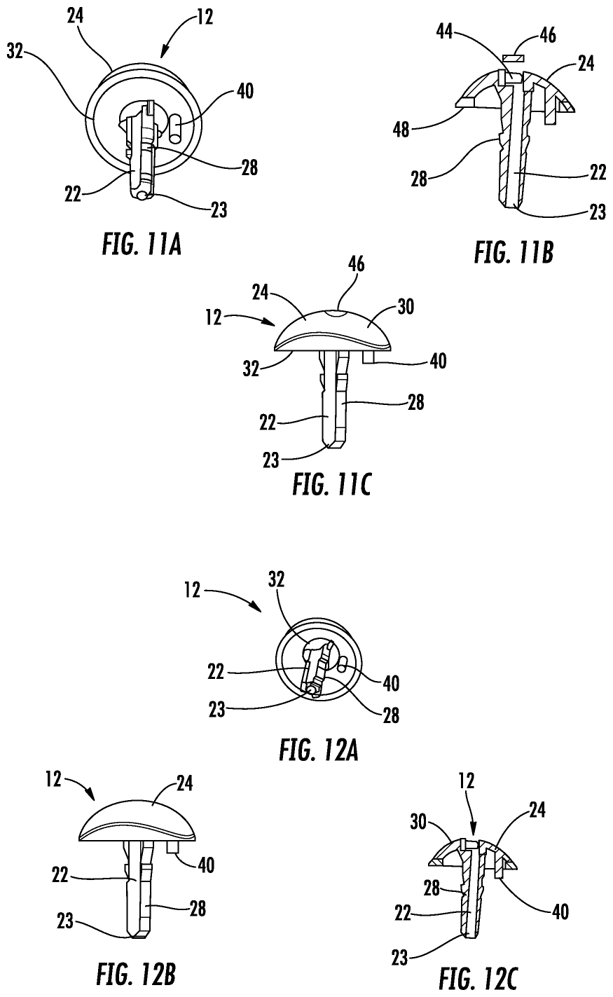

The guide pin is then removed and a trial insertion device corresponding to the selected reamer size is inserted. The trial insertion device includes a small central peg and a top etched marking 44 at the 12 o'clock measurement to ensure that the trial insertion device is properly aligned as shown in FIGS. 11 and 12. The joint is then moved through a full range of motion. 90 degrees of dorsiflexion is desired. If the joint is tight, additional mobilization of the sesamoids can be performed, or additional reaming of the metatarsal head is undertaken. If the joint appears loose, a +2 mm. head can be used as the trial insertion device.

If the implant includes one or more fin members 28, the guide pin is reinserted, and a fin template is placed over the guide pin. The template is oriented, so the laser-etched line sits at 12 o'clock relative to the MT head. The template also preferably has peripheral holes corresponding to the spike members 40. For example, if three spike members are provided, the peripheral holes are oriented at 2, 6 and 10 o'clock to provide for the stabilization of the template, as well as fixation points for the peripheral spike members 40 that serve as additional points of fixation of the implant. These holes are drilled with short 0.045″ Kirschner style wires. The number of peripheral holes will correspond to the number of spike members 40 that are to be used with the implant 12. A drop-in drill sleeve with inner and outer portions is then placed in the raised portion of the fin template. A tapered drill is inserted in the inner sleeve, and drilled to a preferred depth of about 14 mm to create the stem aperture 26. There is preferably a hard stop to prevent over-penetration of the tapered drill. The inner sleeve is then removed, and a broach for the fin members is placed over the guide pin, aligned with the dorsal laser-etched line, and impacted to create a space for the insertion of the fin members 28 of the implant 12 at a later step. The extractor is then used to remove the broach and the template is then removed from the MT head.

The final implant 12, which is separately packaged sterile, is then inserted with an impactor, taking care to orient the implant with the laser-etched line 44 on the implant 12 and positioning the fin members 28 within the previously prepared fin slots, if present, and placement of the spike members 40 into the previously prepared holes. The hallux is placed through the entire range of motion to ensure there is no residual impingement. Fluoroscopic images are obtained in the frontal and lateral planes to verify accurate placement and sizing of the implant 12. If additional space preparation is needed or if the range of motion is limited, the implant 12 includes a pair of laterally spaced notch members 36 that may be accessed to assist in removing the implant 12. This process may be repeated until the surgeon is satisfied with the range of

motion that is present in the metatarsal phalangeal joint 20. Intraoperative fluoroscopy may be repeatedly used to visualize the metatarsal shaft and confirm the final positioning of the implant.

Once the proper positioning and sizing of the implant is confirmed, the capsule is closed with 2-0 absorbable suture, 3-0 (or) 4-0 absorbable suture is used for subcutaneous tissues, and 4-0 nylon for skin closure. A compressive dressing is applied, and the foot placed in a short walking boot. Heel weight bearing is allowed, and range of motion exercises are initiated on day 2. Sutures are removed 10 days after surgery, patients are permitted to bear weight as tolerated, physical therapy initiated, and the patient weans out of the boot as tolerated.

The implant 12 of the present invention preferably includes a range of sizes such as 8, 9, 10, 11, or 12 mm for use with lesser metatarsal heads and 14, 16, 18, 20, 22, 24, 26 mm for use in the first metatarsal phalangeal joint 20. The implant may be made of a variety of standard implant materials. In the preferred form of the present invention, the implant may include a titanium head 24 with a plasma sprayed undersurface and a hydroxyapatite coated stem proximal 22. Alternately, the head may be made of a cobalt chrome with a hydroxyapatite coated stem or a trabecular metal stem. Yet another form of the present invention may include a porous bone contacting surface formed of a material such as a titanium based alloy with a powdered bed fusion process to form various pore sizes on a portion of the cap and the stem. Yet another form of the implant 12 of the present invention may be made using currently available 3D printing processes such that the desired pore sizes may be tightly controlled and tailored to various surface areas of the implant 12. Yet another form of the present implant 12 may include one or more recesses or pore surfaces that include bone growth promoting materials therein or thereon. The preferred pore sizes are in the range of between about 0.5 to 1 mm. As shown in FIG. 7, the preferred dimensions of the implant 12 may include a width of the base of the stem adjacent to the head 24 of between about 3.5 mm to 4.75 mm, more preferably between about 3.5 mm to 4.2 mm with a width of approximately 1.8 mm to about 2.4 mm at the end 23 of the proximal stem 22. The preferred length of the stem 22 is between about 12 mm to 14 mm. The preferred thickness of the head is between about 0.4 mm to 0.6 mm adjacent to the stem and preferably tapers to between about 0.25 mm to 0.035 mm at the periphery. Additionally, the thickness of the head adjacent to the proximal stem 22 is approximately twice the thickness of the head at the periphery. In the embodiments having the spike members, the diameter is preferably about 0.04 mm with a preferred length of approximately 3 mm to 7 mm, with a preferred length of about 3 mm such that the length of the spike members as compared to the length of the stem members is less than 50 percent and more preferably about 25 percent or less while the comparative diameter of the spike member 40 is preferably about 25 percent and more preferably about 10 percent or less than the diameter of the proximal stem 22. These ranges are approximate and will vary depending on the overall size of the implant and are preferably optimized to provide a stable and secure implant that will function for many years. As illustrated above, a preferred configuration of the stem includes a thickness at the distal tip that is approximately one-half of the thickness of the stem adjacent to the head.

FIG. 7 is a side view, partially in cross section showing a tapered proximal stem 22 and illustrating the tapered thickness between the interior and exterior surfaces of the head 24. As shown in FIGS. 8A and 8B, the proximal stem 22 of the present invention may also include a consistent thickness between the base of the stem adjacent to the head to the end 23 of the proximal stem 22. As shown, this embodiment includes a single spike member preferably located at a 6 o'clock orientation. The proximal stem 22 of this embodiment may also include fin members 28 having a reduced outwardly extension the extend near the end 23 of the proximal stem 22 as described with the other embodiments. The preferred thickness is about 4.5 mm with a preferred length of about 14 mm.

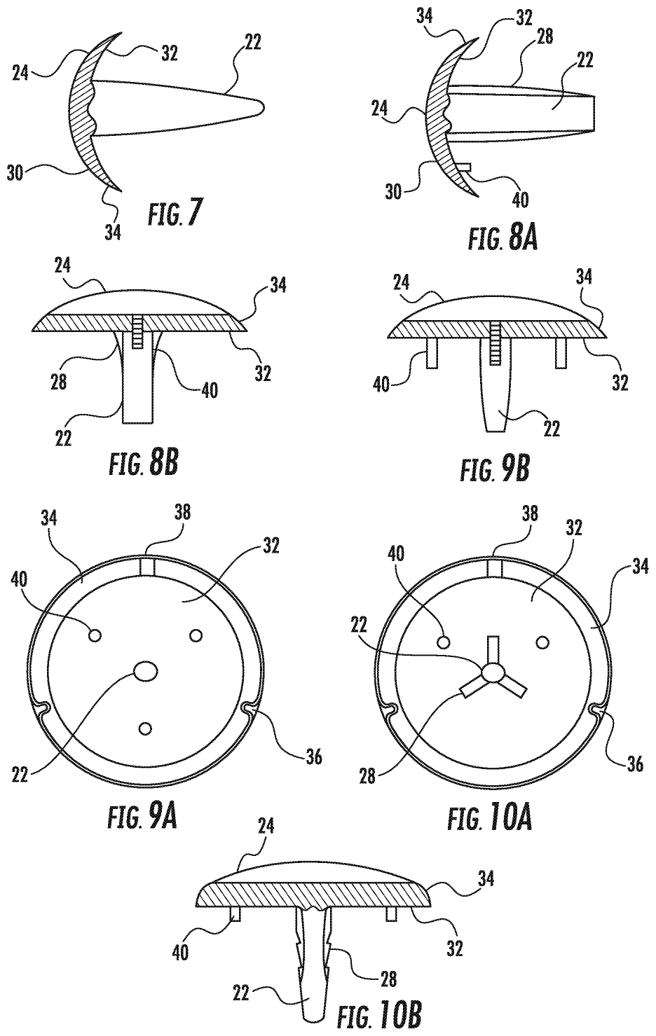

As shown in FIGS. 9A and B, the proximal stem 22 may be a tapered member without fin members. In this embodiment, three spike members 40 are preferably arranged at a preferred orientation of about 2, 6 and 10 and preferably have a greater length than is illustrated with the other embodiments. In this embodiment, the spike members 40 may include a length dimension between about 3 mm to 7 mm with a preferred length of about 6 mm. The proximal stem 22 of this embodiment may be thinner than the other embodiments with a preferred dimension in the range of about 3.5 to 4.2 mm and more preferably about 4 mm.

Another embodiment is shown in FIGS. 10A and 10B, wherein the proximal stem 22 is tapered and includes three fin members 28 oriented at a preferred orientation of about 4, 8 and 12. The fin members 28 of this embodiment are preferably cannulated to provide ridge type surfaces to resist the removal of the fin members 28 and proximal stem 22 from the distal end 16 of the metatarsal bone 14. In this embodiment, a pair of spike members 40 are also utilized. The spike members 40 are oriented at a preferred orientation of about 2 and 10 and are misaligned with the pair of notch members 36 located along the periphery of the implant 12.

Another embodiment is shown in FIGS. 11A, 11B and 11C, wherein the proximal stem 22 is tapered and includes three fin members 28 oriented at a preferred orientation of about 4, 8 and 12. The fin members 28 of this embodiment are preferably cannulated to provide ridge type surfaces to resist the removal of the fin members 28 and proximal stem 22 from the distal end 16 of the metatarsal bone 14. In this embodiment, at least one spike member 40 is also utilized. The spike member 40 are oriented at a preferred orientation which is misaligned with fin members 28 and the pair of notch members 36 located along the periphery of the implant 12. In this embodiment, the greatest thickness between the inner surface 32 and exterior surface 34 of the head 24 of the implant is offset from the alignment of the proximal stem 22. The variation in thickness of the head 24 allows for the further alignment of the area of greater thickness with the desired location of the proximal end 19 of the proximal phalanx bone 18 to form the components of the surgically repaired metatarsal phalangeal joint 20 that more closely mimics the natural metatarsal phalangeal joint. As shown in FIGS. 11B and 11C, this embodiment may also include a threaded inset area 44 that allows the impactor to be threaded into the implant prior to the insertion of the implant 12 into the surgically prepared metatarsal bone. This connection enables the physician to apply direct force to the implant 12 as the implant 12 is inserted into the desired metatarsal bone via a secure connection between the impactor and implant. A removable cap 46 is also shown that may be threaded or also snapped into the inset area 44 to ensure that the outer surface of the head includes the desired exterior surface of the implant. Also shown with this embodiment, the implant 12 may include an outer ledge area 48, preferably oriented at about 9 and 3 to provide a surface area that may be grasped with a removal tool if it is desired to remove the implant from the joint.

FIGS. 12A, 12B and 12C are illustrative of another embodiment wherein the greatest thickness between the inner surface 32 and exterior surface 34 of the head 24 of the implant is offset from the alignment of the proximal stem 22. In this embodiment, the fin members are preferably cannulated and extend outwardly from the proximal stem 22 in a nearly uniform distance with none or only a small amount of reduced width between the extension of the fin member 28 adjacent to the head 24 and end 23 of the proximal stem. Additionally, the spike members 40 of this embodiment includes a greater length and thickness than the spike members 40 described with respect to the other embodiments described herein.

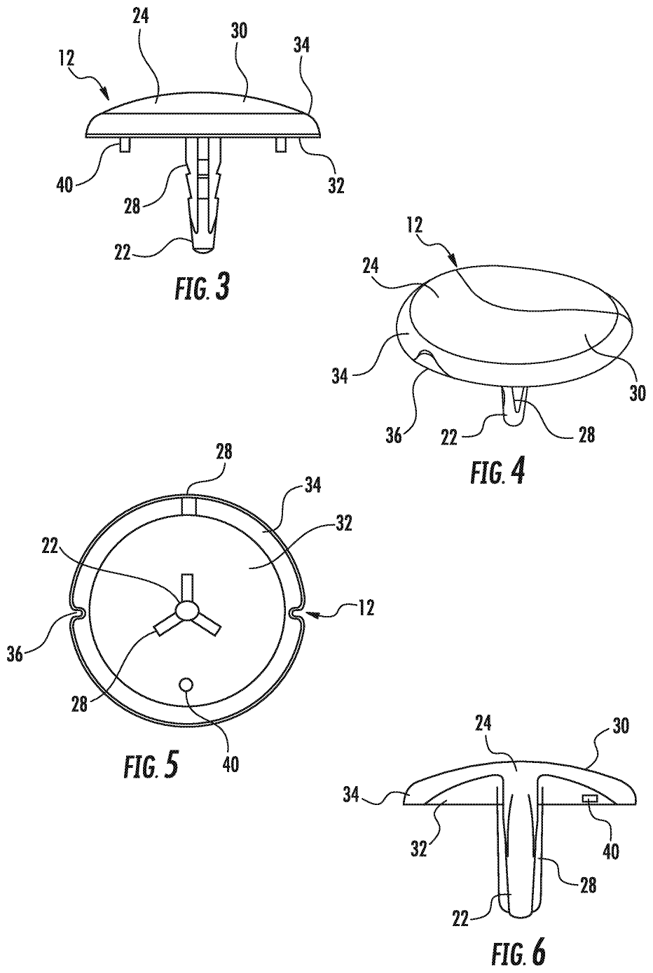

As disclosed herein, the implant 12 of the present invention may include 3 spike members 40 oriented at about 2, 6, and 10 o'clock (FIG. 2, FIGS. 9A and B and 12A and 12B); a single spike member 40 oriented at about 6 o'clock (FIG. 5, FIGS. 8A and B and FIGS. 11A, 11B and 11C); or 2 spike members 40 oriented and 2 and 10 (FIGS. 10A and B). The spike members 40 may also be oriented at different configurations without departing from the scope of the present invention. The implant 12 of the present invention may include various configurations of the proximal stem 22. As shown in FIGS. 2, 3-6 and 10A and B, the proximal stem 22 may be tapered and include a plurality of fin members 28 thereon. As shown, the fin members 28 are preferably tapered along the lengthwise dimension. As shown in FIGS. 7, and 9A and B, the proximal stem 22 may be a tapered member without fin members thereon. As shown in FIG. 8A and B and FIGS. 12A and B, the proximal stem 22 may also have a substantially constant thickness. As with the other embodiments, the combination of the tapered or constant thickness proximal stem 22 with one or more spike members 40 resists rotational movement of the implant 12 when it is implanted and reduces the amount of bone that is harvested during implantation. Additionally, the fin members may be cannulated or non-cannulated.

Although the invention has been herein described in what is perceived to be the most practical and preferred embodiments, it is to be understood that the invention is not intended to be limited to the specific embodiments set forth above. Rather, it is recognized that modifications may be made by one of skill in the art of the invention without departing from the spirit or intent of the invention and, therefore, the invention is to be taken as including all reasonable equivalents to the subject matter of the appended claims and the description of the invention herein.

What is claimed is:

1. An implant for use on a joint-facing end of a bone comprising:
A head member having a convex outer surface overlaying a concave inner surface;
A stem member extending from the inner surface wherein the stem member has a first cross- sectional diameter generally adjacent to the head member and at least one laterally extending fin member thereon; and
A spike member spaced apart from the stem member and extending from the inner surface and wherein the spike member has a first cross-sectional diameter generally adjacent to the head member and the first cross sectional diameter of the spike member is smaller than the first cross sectional diameter of the stem member.

2. The implant of claim 1 wherein the stem member is a tapered member having a plurality of laterally extending fins thereon.

3. The implant of claim 1 wherein a plurality of spaced apart spike members extend from the inner surface of the head member.

4. The implant of claim 3 wherein the plurality of spike members have a first cross sectional diameter generally adjacent to the inner surface of the head member that is smaller than the first cross sectional diameter of the stem member.

5. The implant of claim 3 wherein the plurality of spike members are equidistantly spaced apart from each other and are positioned radially outwardly from the stem member on the inner surface of the head member.

6. The implant of claim 1 wherein the outer convex surface of the implant includes an outer periphery and a plurality of notches that are located along the periphery of the implant.

7. The implant of claim 6 wherein the notches are equidistantly spaced apart from each other along the periphery of the implant.

8. The implant of claim 1 wherein at least one of the concave inner surface and the stem member are formed of a porous material.

9. The implant of claim 1 wherein the head member includes a central surface and a periphery and the thickness of the head member tapers from the central surface to the periphery between the convex outer surface and a concave inner surface thereof.

10. The implant of claim 1 wherein the head member includes a central surface and a periphery and different thicknesses between the convex outer surface and concave inner surface thereof and wherein the head member is thickest between a location between the central surface and periphery.

11. The implant of claim 10 wherein the spiked member is spaced apart from the centrally located stem member on the concave inner surface.

12. An implant for use on a joint-facing end of a bone in a surgical procedure comprising:
A head member having a convex outer surface overlaying a concave inner surface and having an outer periphery thereon;
A stem member extending from the inner surface wherein the stem member has a first cross sectional diameter generally adjacent to the head member that is greater than a second cross sectional diameter that is spaced apart from the inner surface; and
A plurality of notch members located on the periphery of the head member wherein the notched members are spaced apart on the periphery of the head member in a manner to facilitate the removal of the implant in the event of a failure of the surgical procedure.

13. The implant of claim 12 further including a spike member spaced apart from the stem member and extending from the inner surface and wherein the spike member has a first cross sectional diameter generally adjacent to the head member and the first cross sectional diameter of the spike member is smaller than the first cross sectional diameter of the stem member and wherein the spike member includes at least one laterally extending fin member thereon.

14. The implant of claim 12 wherein the stem member is a tapered member having one or more laterally extending fins thereon and wherein the one or more laterally extending fin members extend along the spike member from a location adjacent to the head member to a location spaced apart from the head member.

15. The implant of claim 12 wherein a plurality of spaced apart spike members extend from the inner surface of the head member and the plurality of spike members are equidistantly spaced apart from each other and extend radially outwardly from the stem member.

16. The implant of claim 12 wherein the head member includes a central surface and a periphery and the thickness of the head member tapers from the central surface to the periphery between the convex outer surface and a concave inner surface thereof.

17. A method for using an implant on a joint facing end of a bone, comprising the steps of:
Providing an implant that includes:
A head member having a convex outer surface overlaying a concave inner surface; and
A stem member protruding from the inner surface of the concave inner surface having a first cross sectional diameter generally adjacent to the concave inner surface with a laterally extending fin member thereon and at least one spike member having a first cross sectional diameter generally adjacent to the concave inner surface which is smaller than the first cross sectional diameter of the stem member;
Creating an elongate aperture into the joint facing end of a bone for the insertion of the stem therein;
Inserting the stem into the aperture; and
Pressing the stem into the aperture until the inner surface of the head comes to bear on the end of the bone and the at least one spike member is pressed into the end of the bone. (Previously Presented) The method of claim 17 wherein the head member of the implant includes an outer periphery thereon and the outer periphery includes a plurality of notches thereon to facilitate the removal of the implant from the end of the bone.

19. The method of claim 17 wherein the implant includes a plurality of equidistantly spaced apart spike members thereon and wherein the spike members are pressed into the end of the bone when the stem member is pressed into the aperture.

20. The method of claim 17 wherein the head of the implant is thicker along a portion thereof than along the periphery thereof to allow the surgeon to remove less bone from the metatarsal bone during implantation.
